(19) United States
(12) Patent Application Publication
(10) Pub. No.: US 2007/0086912 A1
(43) Pub. Date: Apr. 19, 2007

(54) ULTRAVIOLET LIGHT EMITTING DIODE SYSTEMS AND METHODS

(75) Inventors: Kevin J. Dowling, Westford, MA (US); Frederick M. Morgan, Quincy, MA (US); George G. Mueller, Boston, MA (US); Ihor A. Lys, Milton, MA (US); Matthew L. Tullman, Lebanon, NH (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: Color Kinetics Incorporated, Boston, MA (US)

(21) Appl. No.: 11/566,381

(22) Filed: Dec. 4, 2006

Related U.S. Application Data
(63) Continuation of application No. 09/923,223, filed on Aug. 6, 2001.
(63) Continuation-in-part of application No. 09/215,624, filed on Dec. 17, 1998, now Pat. No. 6,528,954.
Application No. 09/215,624 is a continuation-in-part of application No. 08/920,156, filed on Aug. 26, 1997, now Pat. No. 6,016,038.
(60) Provisional application No. 60/235,678, filed on Sep. 27, 2000.
(60) Provisional application No. 60/222,847, filed on Aug. 4, 2000.
(60) Provisional application No. 60/071,281, filed on Dec. 17, 1997.
(60) Provisional application No. 60/068,792, filed on Dec. 24, 1997.
(60) Provisional application No. 60/078,861, filed on Mar. 20, 1998.
(60) Provisional application No. 60/079,285, filed on Mar. 25, 1998.
(60) Provisional application No. 60/090,920, filed on Jun. 26, 1998.

(51) Int. Cl. A61L 2/04

(52) U.S. Cl. 422/1

(57) ABSTRACT

Methods and apparatus in which ultraviolet radiation is generated from at least one first LED, an object is irradiated with the ultraviolet radiation, and the ultraviolet radiation is controlled so as to generate at least one perceivable visual effect based on an interaction between the ultraviolet radiation and the object.

### CROSS-REFERENCES TO RELATED APPLICATIONS

This application claims the benefit under 35 U.S.C. §120 as a continuation of U.S. non-provisional application Ser. No. 09/923,223, filed Aug. 6, 2001, entitled “Ultraviolet Light Emitting Diode Systems and Methods.”

Ser. No. 09/923,223 in turn claims the benefit under 35 U.S.C. §119(e) of the following U.S. provisional applications:

Ser. No. 60/235,678, filed Sep. 27, 2000, entitled “Ultraviolet Light Emitting Diode Device;” and

Ser. No. 60/222,847, filed Aug. 4, 2000, entitled “Ultraviolet Light Emitting Diode Device.”

Ser. No. 09/923,223 also claims the benefit under 35 U.S.C. §120 as a continuation-in-part of U.S. non-provisional application Ser. No. 09/215,624, filed Dec. 17, 1998, entitled “Smart Light Bulb,” now U.S. Pat. No. 6,528,954, issued Mar. 4, 2003.

Ser. No. 09/215,624 in turn claims the benefit under 35 U.S.C. §119(e) of the following U.S. Provisional Applications:

Ser. No. 60/071,281, filed Dec. 17, 1997, entitled “Digitally Controlled Light Emitting Diodes Systems and Methods;”

Ser. No. 60/068,792, filed Dec. 24, 1997, entitled “Multi-Color Intelligent Lighting;”

Ser. No. 60/078,861, filed Mar. 20, 1998, entitled “Digital Lighting Systems;”

Ser. No. 60/079,285, filed Mar. 25, 1998, entitled “System and Method for Controlled Illumination;” and

Ser. No. 60/090,920, filed Jun. 26, 1998, entitled “Methods for Software Driven Generation of Multiple Simultaneous High Speed Pulse Width Modulated Signals.”

Each of the foregoing applications is hereby incorporated herein by reference.

### BACKGROUND

1. Field of the Invention

The invention relates to light emitting diode devices. More particularly, the present invention relates to ultraviolet light emitting diode systems and methods for generating ultraviolet light.

2. Description of Related Art

There are many systems that use ultraviolet light. Some systems are designed to generate effects such as fluorescing effects while other systems are used for the purification of objects, liquids and vapor.

Water purification systems are in great demand for industrial, home, portable, and other uses. These systems are designed to purify a predetermined quantity of water before dispensing for consumption or other use. There are many techniques or methods used to purify water. Usually, multiple techniques are employed within one purification device. Filters are generally used to remove particulates from the water while ultra-violet light is used to disinfect the water. The disinfection process may involve passing water through a clear tube while passing ultraviolet light through the tube simultaneously. The ultraviolet radiation is used to eliminate most bacteria and viruses.

There are many known systems for purifying water such as the systems described in U.S. Pat. Nos. 6,080,313; 4,876,014; 5,024,766; 5,190,659; 5,529,689; and 5,573,666. All of these systems utilize mercury vapor discharge lamps to produce the ultraviolet light. These lamps may be high intensity discharge lamps or more commonly low pressure mercury discharge lamps such as fluorescent lamps. These lamps are generally chosen because of their mercury line emission properties. One of the primary resonance lines of mercury is in the ultraviolet at 256 nm. Guidelines from the EPA, EPA Guidance Manual Alternative Disinfectants and Oxidants, April 1999, state that the optimum range for germicidal effects is ultraviolet radiation between 245nm and 285 nm.

There are several problems associated with using high intensity discharge (HID) or low-pressure discharge lamps for the purpose of purifying water. HID sources, for instance, require high voltage and high power sources to operate the lamps. The ballasts for these lamps are large, heavy and not portable. With these constraints, the HID source may provide an acceptable solution for industrial settings but is undesirable for the home or as a portable unit. A problem associated with fluorescent lamps is that the lamps are fragile because they are relatively long tubes of thin glass. This causes a significant problem in portable units because many of these portable units are used while camping or hiking and the units may not be treated with the care required to prevent breakage.

Another problem associated with the use of either low-pressure or high-pressure discharge tubes for the production of ultraviolet radiation is that both of these sources require a significant amount of mercury to produce the desired radiation. Mercury is still a significant environmental and health problem. Many States have regulations covering the disposal of HID and fluorescent lamps and these regulations specify that the lamps cannot go to normal landfills. These lamps must be treated as hazardous waste or be properly recycled to prevent the mercury from being released. Massachusetts, for example, regulates these lamps under 310 CMR 30.000 “Hazardous Waste Management” and Massachusetts DEP Policy “Interim Guidance for the Management of Spent Fluorescent Lamps Containing Mercury.” These lamps must be treated as hazardous waste because of the high mercury content.

There are many other devices that use discharge lamps to generate ultraviolet light. These devices include devices designed for curing ink, cement, glue or enamel (such as the material used by dentists); devices designed to illuminate and inspect articles such as money or other articles with fluorescent properties. In some applications, fluorescent dyes are applied to articles and the ultraviolet source is used to inspect for the presence of the dye.

Ultraviolet Photography is used for many applications to provide information that is otherwise difficult to depict. There are two primary methods of photographing an object under ultraviolet: reflected and fluorescence photography. There are also many applications for ultraviolet photography ranging from recreational, scientific, and medical to geology. Ultraviolet can be used to illuminate objects for inspection as well as illumination for photography to document the appearance of the object under ultraviolet radiation.

Most ultraviolet photography is done by irradiating a photographic sample with long wavelength ultraviolet 320 to 400 nm. Long wavelength ultraviolet is most preferable because most conventional camera lenses pass the long ultraviolet wavelengths. Shorter wavelength ultraviolet can be used but special ultraviolet pass glass must be used in the lens to allow the reflected ultraviolet to reach the film. Another problem associated with shorter wavelength ultraviolet is the dangers to the eyes and skin. When the shorter wavelengths are used, extra precautions must be used, although long wavelength ultraviolet can also pose concern about eye and skin tissue where there are extended periods of exposure. With the proper match between the radiated ultraviolet and the lens that passes these wavelengths the proper selection of film is important. Most black and white film is sensitive to ultraviolet so this is not a problem.

The difference between reflected and fluorescent photography is the method that is used to irradiate the object to be filmed. Normally, fluorescent lamps with special ultraviolet producing phosphors are used to irradiate the object. These lamps emit radiation other than just ultraviolet and this radiation may reside in the visible spectrum. If the visible radiation is not filtered out, the film will react to the visible light and the desired effect may not be achieved. In reflective photography, a filter is placed in front of the camera lens to eliminate all of the visible energy while passing the ultraviolet. In fluorescent photography, an ultraviolet pass filter is placed in front of the ultraviolet source to eliminate all visible energy and an ultraviolet pass filter is placed in front of the camera lens to remove stray light. This technique provides different effects and is useful for several applications including, but not limited to, archaeological photography. See Ultra Violet Photography by Eliadis Elias.

Ultraviolet Photography can be used in many applications such as, but not limited to, capturing images of finger prints, body secretions from animals and humans, images of articles that fluoresce or have fluorescent materials applied, medical images, natural objects, art, repairs made to articles, tracks or residue. When objects are lit with ultraviolet they appear quite different from when they are lit with visible light. Geologists use ultraviolet light to examine stone for composition and identifying materials or the level of water penetration. Ultraviolet sources are required for the inspection of these various items and ultraviolet imagery is required to capture the images.

Another example of ultraviolet inspection or photography is in analyzing insect life patterns. Insects can only see ultraviolet and short wavelength visible light, so the best way to see what they see is to irradiate objects with the same light. After illumination, the best way of capturing these images is to use ultraviolet photography. Many photographers also use ultraviolet photographic techniques for artistic reasons to capture unique images that cannot be achieved with any other technique.

Another area where ultraviolet light is used is in light therapy. Light therapy can take many forms and can be designed to remedy mental, emotional, or physical illnesses or disorders. Full spectrum lighting or specific wavelengths of visible, ultraviolet, or infrared radiation can be used during treatments of such illnesses or disorders. Light therapy has been a valued therapeutic technique throughout history. The sun is a good source of full spectrum lighting and can provide healing effects. Many of the light therapy techniques attempt to provide light that emulates sunlight.

Several recent studies suggest there is an importance in being exposed to full spectrum lighting. According to photobiologist John Nash Ott D.Sc. (hon.), poor lighting can pose a serious threat to health. Most artificial lighting systems, such as incandescent and fluorescent, lack the complete balance of emitted wavelengths to be categorized as full spectrum lights. When 90% of a person's day is spent under these types of light sources, it can affect the bodies' optimal absorption of nutrients. This can lead to many problems ranging from fatigue to depression, even physical ailments. The U.S. Navy recently completed a study of the effect of a person's occupation and its effect on the development of melanoma. The study suggested a correlation between the occupation and the melanoma rate that is counter-intuitive. The occupations that required almost exclusively indoor activities showed the highest incidence rate of the cancer while occupations requiring some outdoor exposure resulted in the least development. This study suggests that some forms of cancer can be treated or prevented by exposure to full spectrum lighting.

### SUMMARY

The present invention relates to ultraviolet systems and methods of producing such systems with light emitting diodes.

An embodiment of the present invention is a purification device. The purification device may comprise a purification chamber, and at least one LED that produces ultraviolet light, wherein the at least one LED is arranged to irradiate the inside of the chamber.

Another embodiment of the present invention is a handheld device. The hand held device may comprise a handheld housing, and at least one LED that produces ultraviolet, light wherein the at least one LED is arranged to irradiate from the housing.

A further embodiment of the present invention is an insect light. The insect light may comprise at least one of an ultraviolet light producing LED and a blue light producing LED for attracting insects, and at least one of an insect trap and insect killing device.

Another embodiment of the present invention is a method of purifying. The method may involve the steps of providing at least one LED that produces ultraviolet light; providing a chamber for containing at least one of a liquid and a vapor; and irradiating the interior of the chamber with the at least one LED.

A further embodiment of the present invention is a method of purifying a surface. The method may comprise providing a handheld housing; providing at least one LED that produces ultraviolet light wherein the at least one LED is associated with the housing and arranged to irradiate from the housing; and having a user hold the housing and irradiate a surface to be purified.

A further embodiment of the present invention is a method of irradiating an object with ultraviolet light. The method may comprise providing a handheld housing; providing at least one LED that produces ultraviolet light wherein the at least one LED is associated with the housing and arranged to irradiate from the housing; and having a user hold the housing an irradiate an object.

Another embodiment of the present invention is directed to an illumination device. The illumination device may comprise at least one visible LED that generates visible light, at least one ultraviolet LED that generates ultraviolet light, a processor that independently controls the at least one visible LED and the at least one ultraviolet LED, and a housing wherein the LEDs are housed and arranged to irradiate from the housing.

Another embodiment of the present invention is directed to a method of irradiating a display. The method may comprise the acts of providing a display, providing a plurality of ultraviolet LEDs, and irradiating the display with the ultraviolet LEDs.

Another embodiment of the present invention is directed to a method of impacting the growth of plants. The method may comprise the acts of providing at least one ultraviolet LED, providing at least one visible LED, providing a processor that independently controls the at least one ultraviolet LED and the at least one visible LED, directing the at least one ultraviolet LED and the at least one visible LED to irradiate a plant, and causing the processor to vary the output of the LEDs over a period of time.

### BRIEF DESCRIPTION OF THE FIGURES

The following figures depict certain illustrative embodiments of the invention in which like reference numerals refer to like elements. These depicted embodiments are to be understood as illustrative of the invention and not as limiting in any way.

FIG. 1 illustrates a purification device according to one embodiment of the present invention.

FIG. 2 shows a stand-alone LED unit for inspection or for ultraviolet photography according to another embodiment of the present invention.

FIG. 3 shows two LED units according to another embodiment of the present invention with one combined into the front of a camera and one located on the top of the camera as a detachable unit.

FIG. 4 illustrates an ultraviolet inspection device with a magnifying glass according to another embodiment of the present invention.

FIG. 5 illustrates a flashlight style ultraviolet source according to a further embodiment of the present invention.

FIG. 6 illustrates an ultraviolet source according to another embodiment of the present invention.

### DETAILED DESCRIPTION

The description below pertains to several illustrative embodiments of the present invention. Many variations of the invention may be envisioned by one skilled in the art. Such variations and improvements are intended to fall within the compass of this disclosure. Thus, the scope of the invention is not to be limited in any way by the disclosure below.

One embodiment of the invention is directed to the use of ultraviolet radiation, generated light emitting diodes, for purification, inspection and many other uses. This provides a number of advantages over conventional UV sources, including that it is mercury-free.

The advent of the high brightness light emitting diode (LED) has opened up many new applications for the LED. The LED was primarily used as an indicator light and now is being used as an illumination device. The brightness of the LED has been increasing exponentially over the past three decades. LEDs are now being used in color changing illumination devices such as that described in U.S. Pat. No. 6,016,038. LED manufacturers such as Nichia, Lumileds, Philips, Siemens and Osram Opto are all attempting to create highly efficient high quality white light producing LEDs. This is for general lighting applications to replace incandescent, halogen, and fluorescent lighting.

White LEDs are generally devices that produce blue, violet or ultraviolet light, which is then converted to visible radiation through a phosphor. If the phosphor layer is eliminated, the LED becomes an ultraviolet radiation source. Nichia has also recently announced a violet LED where the primary emission spectrum has wavelengths between 395 nm and 420 nm. This short wavelength may be acceptable for water purification purposes. If even shorter wavelengths are desired, U.S. Pat. No. 6,084,250 discloses an LED with emission centered between 300 nm and 370 nm. Other ultraviolet producing LEDs are available or can be manufactured to produce different ultraviolet bands of radiation. The recent trends in the development of ultraviolet LEDs indicate that even shorter wavelength producing light emitting diodes will soon be available. A die could also be developed to produce deep ultraviolet for the production of ozone to assist in the water purification process. Ozone treatment is typically a separate process from ultraviolet treatment.

The ultraviolet-producing LED can be used to purify water in a similar fashion as the mercury-containing discharge tube methods. The purification system can include one or more LEDs to provide the requisite level of ultraviolet radiation. This new method of water purification can be provided as a stand-alone device or in combination with other purification devices such as, but not limited to, filters, scrubbers, other ultraviolet sources, mixers and any delivering system.

An ultraviolet-producing LED device can also be provided to kill bacteria and viruses or for general sterilization in non-water applications such as for the treatment of surfaces, tables, countertops, walls, floors, ceilings, instruments, tools, utensils, storage units, food handling devices, food, drinks or any other surface, device or object that can be sterilized. One example of using the device in a non-water application is where a countertop in a kitchen needs disinfection. A handheld LED device can be used by sweeping it across the counter in the same way a sponge is used. The device could also be designed to automatically cover or cross over the counter. Another application would be where the device is combined with a medical instrument drawer. The device could be arranged to irradiate the inside of the drawer to provide sterilized instruments. This device could also be used in conjunction with or following other sterilization procedures. One significant problem with sterilization is keeping the objects sterile after the sterilization process is complete. So, in the example of medical instruments, the instruments could be exposed to ultraviolet radiation in a storage tray or drawer following any other sterilization process as a method of maintaining the instruments' sterile condition.

The UV-producing LED system can take any of numerous forms, and is not limited to any particular implementation. For example, in one embodiment, the LED system can have one or more LEDs arranged into a fixture. The LEDs can be controlled by passive or active circuitry. The power to the LED can be controlled through current regulation, voltage regulation, waveform modifications, or other regulation or modulation techniques. The waveform modification can take the form of a pulse width modulated (PWM) waveform signal processing. The PWM control could take any number of forms to produce any number of functions such as, but not limited to, power source conservation or maximizing, optimizing the efficiency of the process or other functions. Whether passively controlled or actively controlled, each LED could be controlled independently or as a group.

In one embodiment, a microprocessor can be used to regulate the LEDs. The microprocessor can control individual LEDs or a group of LEDs. The microprocessor could have a number of predefined control signals that could be sent to the LEDs. The microprocessor could also have one or more programming devices to provide an input signal. The control signals could be generated and/or communicated in response to the input signals. The programming device could be connected to one or more potentiometers, switches, transducers, sensors or other devices or combinations of devices. When the programming device is activated, changed or sends a signal, a controller may react by sending control signals to the LEDs.

One example of using a programming device in the water purification system is where optical feedback is desired. Generally, the water should be exposed for a certain amount of time under a certain amount of energy. An energy detector can be employed as the programming device to monitor the energy output and adjust the control signal if the output has changed. If the energy falls below some predetermined acceptable level, the device can indicate a problem. This leads to another advantage of an LED device that includes multiple LEDs, because a single LED failure does not render the system incapable of operation. The system can continue to operate and provide purified water with one or more LED failures. If a feedback system is also used in the device, the energy for the remaining diodes could be increased to compensate for the lack of system power.

In one embodiment, several LEDs (two or more) with different outputs could also be used in this system. The different LEDs could be independently controlled or controlled as a group. This system could be used to optimize the purification process. One or more of the LEDs could produce visible light to make indications of certain conditions. If a water purity sensor is connected to a programming means, the visible light LEDs could be activated to indicate the process is complete or at what stage the process is in or what mode the system is in. Two or more colored LEDs could be used to produce different colored outputs. One of the LEDs could also be used as a transmitter to provide communication to other devices. The visible LEDs could be activated to produce visual effects to provide an indication of the device's operating mode or to provide information or for aesthetic reasons. There could be any number of lighting effects produced by a single or multiple LEDs in combination. These could be effects such as color-changing, fixed colors, pulsing colors, strobing colors or any other effect. The lighting effects could also be initiated from another device through communications means.

In one embodiment, a programming device could be connected to other sensors for electromagnetic signal reception to allow the programming device to receive information from external sources or other components of the purification system. Other types of transmitters could also be controlled to allow communication from the water purification system. With receivers and or transmitters and or physical connections, the system can be part of a network. As such, the system could listen for instructions by listening for its particular address and the system could react to the instructions. For example, there could be a water monitoring device or system analyzing the purity of the upstream or downstream water and the monitoring system could change the control signals to change the irradiation level.

A water purification system such as that described could be used to purify water from any source, including purification of water from a fish tank, pond, swimming pool, fountain, spa, or other water source.

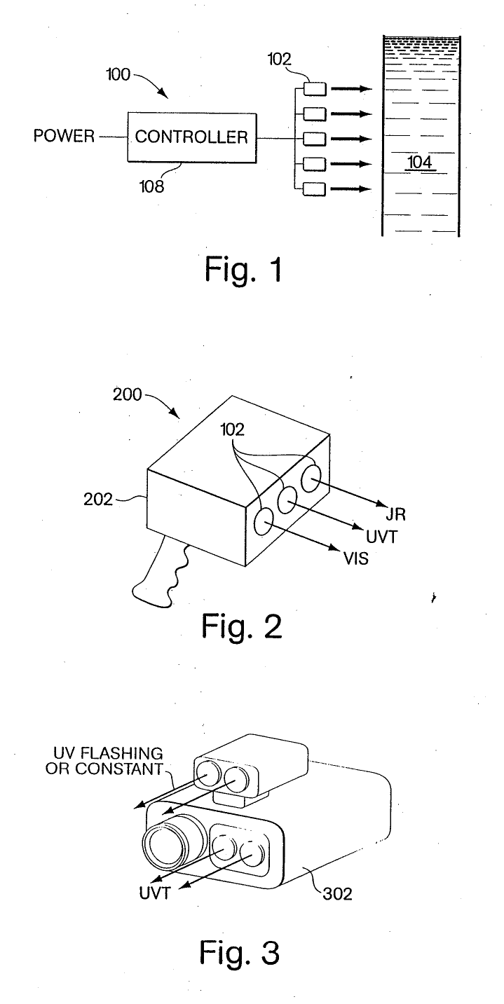

FIG. 1 illustrates a purification system according to one embodiment of the present invention. LEDs 102 are arranged to irradiate chamber 104, which contains water, from one side but the arrangement of LEDs 102 could take many different forms. For example, the LEDs 102 may be arranged within the chamber or external to the chamber. When the LEDs are arranged external to the chamber, a material may be incorporated into the chamber to provide for the transmission of the ultraviolet light. In this embodiment, the LEDs 102 are being controlled by a controller 108. The LEDs 102 do not need to be provided on the outer perimeter, they could also be mounted on the inner perimeter or inside of the water stream or bath. The water could be in a stagnant, agitated or mixed bath or could be flowing during the irradiation process. The LEDs can be arranged in any manner with respect to the water and the water can be presented in any manner so long as the water is irradiated. Although FIG. 1 illustrates the chamber 104 as containing a liquid, the chamber could also be arranged to contain a vapor or a solid.

Another embodiment of the present invention is directed to an ultraviolet LED device for ultraviolet photography, inspection, or detection.

In one embodiment, an ultraviolet radiation device is provided where the primary, secondary or only source of ultraviolet radiation is an LED device. This device can be used as a stand-alone device or in combination with other devices or combined with a network to be a network device. In a network arrangement, the controller 108 may be an addressable controller.

A device according to the principles of the present invention may also be incorporated into a still frame camera, motion picture camera, video recoding device, or other recoding device. The photography systems can use film as the recording media or they can store the images digitally or by any other means.

In one embodiment, the ultraviolet radiating device may be used as a stand-alone device for inspection, irradiating, or detection purposes. This device can be used for any purpose where ultraviolet irradiation is desired. Such uses include, but are not limited to, inspection of materials, body secretions, fluorescent display or analysis, or medical reasons. The device can also be incorporated into other systems such as manufacturing lines and process control where ultraviolet light is used for imaging and or control.

In one embodiment, a device may be constructed with one or more LEDs with different output spectra to provide the desired radiation output. Several different LEDs with different wavelength characteristics could also be used for various applications. One example of using different wavelength LEDs is where one ultraviolet radiating type of LED is combined with another ultraviolet radiation LED of a different ultraviolet wavelength or where visible or infrared (IR) LEDs are combined in the system. Visible LEDs may be combined for effect or for assisting the user in aiming the radiation towards the subject or object to be photographed. Output levels can be adjusted once the illumination direction and pattern are set. The ratio of ultraviolet to visible to IR emission properties of the device could also be changed to suit to a particular application.

There are many materials available that fluoresce when irradiated with ultraviolet light or deep blue light. In one embodiment of the invention, any of these materials can be used in signs and displays in conjunction with an LED light to create unique visual effects. The materials include, but are not limited to, plastics, such as core, rod, tube and sheet; paints and dyes such as fluorescent, phosphorescent and invisible paint that is revealing under ultraviolet or blue irradiation; water dyes; bubble fluid; and any other material that reflects, fluoresces or phosphoresces. Applications where these materials can be used with LED lighting include, but are not limited to, displays, identifying marks, backdrops, scenic artwork, body paints, tattoos, club wear, party products, bar products, catering products, 3D glasses, floor tiles, special effects in movies, films, television, theater, concerts, events, conferences, press launches. Other applications include, but are not limited to, using these materials and LED lighting for scenic effects at clubs, pubs, bars, cinemas, casinos, hotels, theme parks, amusement park attractions, bowling alleys, quasar arenas, amusement arcades, and any other area. Other applications include, but are not limited to, promotion and advertising at the point of sale, in window displays, signs, billboards, exhibition stands, and product launches and any other display.

FIG. 2 shows a stand-alone LED device 200 according to one embodiment of the present invention for purification, inspection, ultraviolet photography or other uses. The device 200 in this embodiment may include one or more LEDs 102 and a handheld housing 202. As indicated in FIG. 2, the device may include more than one LED with different spectral output. FIG. 3 shows another embodiment that includes two LED units, with one combined into the front of a camera 302 and one located on the top of the camera as a detachable unit.

The LEDs produce light almost instantaneously upon the application of the control signal and this makes them suitable for a flashing or pulsing mode to create different effects or power supply conservation. The device could be set to pulse periodically while the subject is rotated or moved or the pulse could be applied much like a regular camera flash where the application or radiation only occurs at the moment of the shutter opening.

As mentioned above, the LEDs in a device according to the embodiments of the invention can be controlled by passive or active circuitry. A microprocessor could be used to provide control signals to the LEDs or network of LEDs. The microprocessor could also have an input signal from a programming device. The programming device can include a receiver for the receipt of an input signal from another device for example. The input signal could come from a camera or other device such as, but not limited to, a transducer, switch, transmitter, or other device to supply a signal. The signal could be received digitally or as an analog signal through an A/D converter. The controller can also be connected to a transmitter or other output device to provide communication with other devices. The LEDs can also act as communication devices whether the ultraviolet, visible or IR LEDs are used. A PWM control signal can modulate light control output while making communication output on the same or separate LED. The LED reacts so quickly to the drive signal that one LED can be providing both communications and illumination simultaneously. Separate LEDs can also be used to provide communications.

Pulse width modulated (PWM) control signals, as defined in U.S. Pat. No. 6,016,038, which is hereby incorporated by reference herein, could be used to drive the LEDs where the control signals correspond to an input signal to change the mode of operation. This technique could be used to modulate and thus regulate the output of any of the LEDs. The combination of ultraviolet to visible to IR can be varied to obtain a large range of effects.

In one embodiment, visible LEDs are used in the device. The visible-light producing LEDs may be used, for example, as a reference for irradiation direction and/or intensity. Generally, the ultraviolet photography techniques require trial and error to determine the proper exposure time for a given setting and object. Ultraviolet meters can also be used with the device but there are no good correlation coefficients determined for ultraviolet photography as in visible light photography. However, an ultraviolet meter could be combined with this device to provide feedback. Another method of feedback would be through an LED power meter or control signal indicator. The visible LEDs could also be used to reference how much ultraviolet radiation there is. Because the ultraviolet radiation is not visible to the user, the user does not have feedback as to the light intensity. A correlation, whether theoretically or empirically determined, could be drawn between the visible light intensity from the visible LED and the ultraviolet LEDs. This would allow the user to regulate the intensity of the visible light to set the ultraviolet and then turn the visible LEDs off or reduce their input. This could be useful to a user in setting or adjusting the intensities required to make some effects.

The microprocessor could have a look-up table or a function that equates the visible light intensity to the ultraviolet light intensity. This table or function could also be user adjustable to provide a customized calibration solution.

A device according to the principles of the present invention can also be used for viewing and inspection of objects without photography. In one embodiment, a device is presented incorporating inspection optics to aid the inspection process. The optics could include any optics such as, but not limited to, magnifying glasses or microscopes. Such a device could take various forms and be portable or non-portable. This type of device can also be incorporated into other systems for inspection. The device could be incorporated into vision systems where the objects in the inspection areas are better defined under ultraviolet.

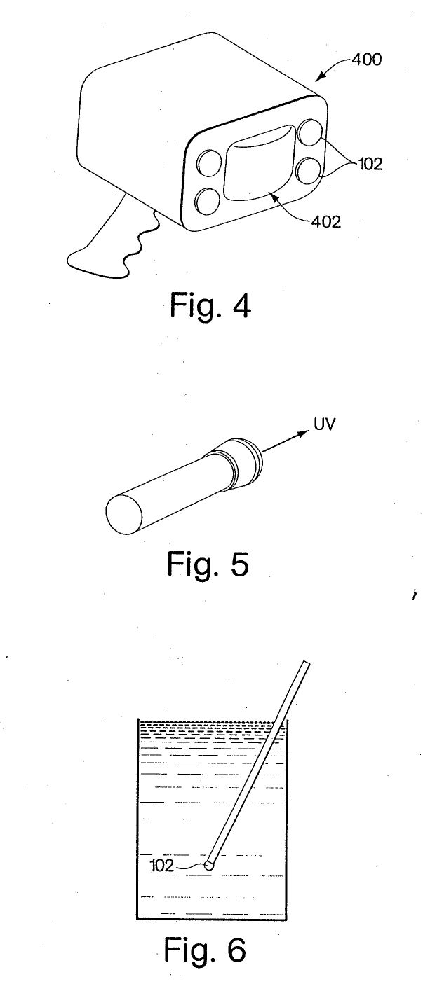

FIG. 4 illustrates an ultraviolet inspection device 400 according to one embodiment of the present invention, with a magnifying glass 402 and LEDs 102. FIG. 5 illustrates a flashlight style ultraviolet source according to another embodiment.

Some of the inspection and photography applications include, but are not limited to, lithic sourcing such as a tool for the inspection of stone, identification of money, identification of stamps and dyes, examination of articles that have been repaired, glue, adhesive, epoxy, oil, grease, in conjunction with rodent control where the body excretions of the animals leave traces that fluoresce, medical examinations, laboratory testing, fluorescent liquid penetrent detection, arson detection, identification of ultraviolet sprays, finger print identification, and other surfaces or articles that are useful to inspect and photograph with the aid of ultraviolet.

A device as described herein may also be used as a curing system for inks, cements, enamels, epoxies, or any other material that can cure under ultraviolet radiation. The device can also be used for black lights, sun tanning, EPROM erasure, web printing, air purification or sterilization of materials. A curing device, inspection device or other device using LED-driven ultraviolet sources can also be coupled with ultraviolet passing fiber optics to provide local or distributed ultraviolet radiation to a remote area. The output from the fiber optics can be connected to other optics for further distribution of the light.

Localized tanning, or tanning a pattern on skin, may also be achieved by using a system according to the principles of the present invention. The LED light sources are compact enough that a single LED can be slowly moved over the skin to provide localized tanning. Similarly, a group of LEDs could be formed into a pattern and used to irradiate the skin with a pattern. By varying the beam angle of the LED, the pattern size and definition could be changed. A group of LEDs could also be assembled in a lighting fixture with optics to provide focusing or a pattern of light to create the tanning pattern. The optics could also include fiber optics to provide remote access. An LED device could be incorporated into clothing to provide irradiation while the clothing is worn.

One especially effective application of ultraviolet irradiation for germicidal effects has been in the control of microbial growth in air handling systems. Legionnaire's disease can be caused by bacteria or fungi found in a building's air handling system or near outdoor air intakes. In particular, the constant exposure of the cooling coil and filter assembly to ultraviolet has been found to be very effective at controlling fungal growth. Viruses are especially susceptible to ultraviolet, more so than bacteria. Viruses are more sensitive to wavelengths above the mercury emission 254 nm. See Aerobiological Engineering Ultraviolet Germicidal Irradiation, www.engr.psu.edu/ae/wjk/wjkuvgi.html. In one embodiment, a UV LED device may be arranged to irradiate an air chamber and or a filtration system within an air handling system to purify the air and/or the handling and/or filtration system.

In one embodiment, an LED light fixture can be incorporated into automotive dashboard lighting, mirror lighting, or any other area within or outside of the automobile, or for other sign and display applications. Light piping or edge lighting can be combined with phosphorous or luminous materials such that they fluoresce when the LEDs are activated. The luminous material could be applied as a layer to provide surface lighting or it could be applied in a pattern.

Plants require light to grow and there are many artificial light sources that are designed to irradiate plants where there is a lack of sunlight. These systems use HID, fluorescent, and incandescent light sources to provide the requisite light. There have been studies of the effect of using red LEDs alone or in combination with fluorescent lighting that shows some positive effects on plant growth. See Light-Emitting Diodes for Plant Growth, W. M. Knott, Ph.D., and R. M. Wheeler, Ph.D., MD-RES, http://technology.ksc.nasa.gov/ WWWaccess/ techreports/94report/lsf/ls04.html. There have also been studies showing the effects that various ratios of different wavelengths have on plant growth behavior. See Effects of Various Radiant Sources on Plant Growth, Shinji TAZAWA, Light Source Division, Iwasaki Electric Co., Ltd, http://ss.jircas.affrc.go.jp/engpage/jarq/33-3/tazawa2/tazawa2.htm and Plant Growth and Development, USDA NRICGP Abstracts of Funded Research, FY 1997, http://www.reeusda.gov/crgam/nri/pubs/archive/abstracts/abstract97/plgrwdev.htm. There have also been studies of interrupting the light cycle and its affects on flowering. See A Review of Factors Affecting Plant Growth, Marianne Ames, Graduate Fellow Wayne S. Johnson, Assistant Professor University of Nevada, Reno, http://www.hydrofarm.com/content/ articles/factors_plant.html. Lighting can be used to slow or increase plant growth.

Systems and methods according to the present invention may also be used for plant growth control. This type of lighting system could be used indoors or outdoors as a plant growth inhibitor or a plant growth aid. The LED device can be arranged with one wavelength LED or several wavelength LEDs covering the ultraviolet, visible or IR. By using the controlling techniques described in this disclosure, ratios of light could easily be produced and customized for particular uses. For example, if the desired output requires a higher blue-to-green ratio, the intensity of the blue LEDs could be increased and/or the intensity of the green LEDs could be decreased. This type of spectral manipulation could be controlled with this LED system. The lights could also be programmed to change the LED outputs as a function of time or other input. For example, if a photocell is used as a programming device, the lighting device could increase its overall output to compensate for the lack of sunlight at any given time. An LED-based device could be designed to adjust its output if the plant requires more ultraviolet or more IR or a particular ratio of light at a particular time of day or cycled throughout the day. These dynamic color and radiation changing effects can provide for many opportunities for enhanced plant growth or reduced plant growth.

In one embodiment, the lighting device may be programmed to simulate normal exterior lighting conditions in areas where sunlight is minimal or not available. The simulation could also be adjusted to enhance or reduce growth. For example, the spectra from the system may be modulated and/or the timing of the cycle may be changed. In one embodiment, a program may be designed to simulate the daylight cycle over the period of 24 hours and the program may be modified to accelerate the cycle such that more or less simulated cycles are performed in a 24 hour period. Cycling the day's simulation multiple times within a 24 hour period, for example, may enhance the growth of plants.

Systems and methods according to the present invention may also be used to provide full spectrum or partial spectrum lighting for general illumination, therapy, treatment, special illumination conditions, tanning, or any other lighting situation where full spectrum or selective spectrum lighting is desired or required. These devices can be made or designed for specific applications or for general applications. Devices made for general applications can also be adjustable to tailor the device to a particular need. The LED device could be made with several different wavelengths producing LEDs or one particular wavelength or wavelength region. Ultraviolet, visible or IR producing LEDs could be employed and several LEDs from each of these spectral regions could be employed. Each of the selected wavelengths or spectral regions or wavelengths within the spectral regions could be varied in intensity by using a greater number of the specific LEDs, or greater intensity LEDs within the desired range, or by controlling all or some of the LEDs to provide variable output control.

In one embodiment, many UV-emitting LED lighting devices or a single device may be used in an office or room or outdoors for the general purpose of providing full or partial spectrum lighting as well as general lighting of the area or objects. The device could also be used in a therapy or medical setting in conjunction with therapy or medical techniques.

In one embodiment, an application may include an LED device used for lighting an office environment and the color temperature of the light as well as the ultraviolet and infrared components of the emission change to simulate the outdoor conditions corresponding to the hour of the day. This could also be used to artificially simulate the wrong hour of the day such as when people are working night hours and they could be exposed to daylight lighting conditions during the work hours to keep their internal clock in synch with their working hours. Early morning hours could have a relatively low ultraviolet component with a low color temperature and some infrared light while midday light levels would have a higher color temperature with elevated levels of ultraviolet. The ultraviolet, or any other wavelength, can be selectively excluded to avoid problems. For example, the ultraviolet spectrum is broken down into three categories, UVA, UVB, and UVC. UVB and UVC are frequently associated with causing skin and eye irritation within relatively short exposure times so these wavelengths could be eliminated or reduced in output to prevent over-exposure. There may also be applications where the deeper ultraviolet wavelengths are desired and could be included or increased in intensity.

An LED device according to the principles of the present invention could also be very versatile in what wavelengths it emits and at what intensity it emits. Each energy region could be selectable and adjustable to allow a user to make the required or desired adjustments to suit the particular application. The device can also be programmed to go through any cycle. A doctor may prescribe a bright light treatment of 15 minutes where the visible light intensity is high but the ultraviolet light intensity is low followed by a 10-minute period of low visible energy but higher ultraviolet or IR. The device could also be adjusted through switches, a single switch, transducers, receivers, detectors or any other device to provide an input signal.

A full spectrum or partial spectrum LED device could also be used for product testing. Many products are designed to be used outdoors where they are exposed to various lighting conditions. Testing conditions are often difficult to reproduce in the laboratory and specific lighting conditions are some of the effects that are difficult to reproduce. For example, a test could be devised using the LED device to simulate a summer day in Nevada and the product could be tested under that simulated light. The LED device could be used in conjunction with other testing elements to create various conditions. For example, the lighting device could be combined with an oven to simulate the heat and lighting effects of the Nevada summer day. Once these conditions are simulated the user can subject the products to continuous or varying conditions as a way of accelerating the testing.

These LED devices can also be used to treat or prevent physical illnesses. Psoriasis and Jaundice are two medical conditions that are normally treated with the application of ultraviolet light. Ultraviolet sources are also used to irradiate blood for treatment of disease and other blood borne viruses including HIV. The LED device can also be used for irradiating tissue or organs in a medical setting for identification or therapy.

In one embodiment, a light-emitting diode based ultraviolet light source could be located in the front of a vehicle. This could be useful in illuminating the lines on the roadway surface. Highway lines, for example, are typically painted white lines and will fluoresce if illuminated with ultraviolet light. The paint could also be enhanced to optimize or increase the fluorescing effect. By providing this type of illumination, the lines on the road would be much more pronounced as compared to the same roadway lit with halogen lamps. The vehicle could be any type of vehicle such as, but not limited to, an automobile, car, motorized vehicle, non-motorized vehicle, bicycle, motorcycle, moped, truck, buggy, or a bus. Optics can also be used to focus the light at a set distance. This could be used to provide high intensity of the light on the roadway line. The beam could be focused to a point or spread over an area.

The LED light source could be equipped with LEDs of a single color such as ultraviolet or the light source could have a combination of several colors. A combination of blue and ultraviolet may be appropriate to provide an indicator that the light is on. The blue emitters would indicate that the light was energized while the ultraviolet emitters would illuminate the road to cause the fluorescing effects. Any combination of different wavelength emitters could be used. Traditionally, yellow driving lights have been used in the front of cars as fog lights because the longer wavelength yellow light scatters less in the fog than lights producing a significant blue component. The other reason for using yellow light is that yellow is near the center of the eye's photopic sensitivity curve, so yellow light is more efficient. With this invention, ultraviolet emitters could be combined with yellow emitters to provide visibility and fluorescing effects. Single color yellow, white or other colors may also be used in the vehicle to produce desired illumination.

An advantage of using ultraviolet LEDs as a secondary illumination source on the front of an automobile is that the ultraviolet can be directed towards the lines on the road, away from other vehicles or pedestrians. This can help alleviate any problems associated with directing the ultraviolet radiation directly at such targets. This device would be relatively simple as compared to the alternative for ultraviolet generation on an automobile. The only other realistic alternative is a discharge light source. These sources use high cost electronics for proper operation and they are generally wide band emitters. With the LED device, the separate colors used could also be individually controlled to change the radiation output of the device. This may be useful for certain driving conditions, as an aid to people with particular vision impairments, or as a decorative element of the car. The lights could also be dimmed and color tuned to make the car more attractive. In another embodiment, the UV emitters may only be activated once the vehicle has achieved a predetermined speed.

An LED lighting device on an auto could also be used as a communication device. The LEDs respond almost instantaneously to the application of power and provide for an excellent communications device. IR LEDs are typically used in remote control devices because of these properties. The LED device could also be used in tollbooths, gasoline stations, service stations, convenience stores, or other venues in the identification of automobiles or other application.

An ultraviolet LED system according to the present invention can also be used for pasteurization. Normally, a thermal process is used to accomplish pasteurization but the thermal processing units are large and expensive to purchase. There are many fruit juice producers and milk producers that operate small businesses or limited production. These operators could benefit by using an LED device which could be smaller, lower cost and easy to operate. Ultraviolet radiation has proven to be an effective method for reducing or nearly eliminating the bacteria E. coli in fruit juices and ciders. See www.sciencedaily.com/releases/1998/01/980127065910.htm Pasteurized via Ultraviolet Light Could Zap Bacterial Contamination of Fresh Cider and Fruit Juices.

Applications where this type of ultraviolet irradiation device would be useful as a disinfection device include, but are not limited to, drinking water, waste water, beverages, spring water, cooling towers, hydroponics, waterfalls and fountains, swimming pools, hydrotherapy, pools, spas, hospital or laboratory water, pharmaceutical manufacturing, pre reverse osmosis water disinfection, food and drink processing, aquarium and fish hatcheries, purification of oysters, any optically transparent liquid, white vinegar, apple cider, organic cutting oils, warm water loops, fish farming, agriculture, and aquariums. Another use in the pharmaceutical manufacturing process is to cure the coatings on tablets.

Ultraviolet light is also used in laboratories as a method of breaking the bonds of chelating agents. To facilitate this reaction an LED ultraviolet device according to the present invention can be provided. The device could be much like a swizzle stick, as indicated in FIG. 6, where at least one ultraviolet producing LED 102 could be included in the end of the stick that is dipped into the liquid. This style of ultraviolet device can also be used for purification of individual containers of liquid. Several LEDs could be included to increase the ultraviolet radiation or add color to the liquid or container. In one embodiment, the UV device may be in the shape, or have a housing in the shape, of an ice cube. As with all of the other devices described herein, the LED can be driven with control signals from a processor or the LED can be driven with passive circuitry. The LED circuitry can simply turn the LED on and off or power regulation of the different LEDs could be employed. If regulation is desired, it can be accomplished through passive circuitry or controlled through pulse width modulation current control or any other control method.

Another application for the LED device is in a spectrophotometer. Spectrophotometers are analytical tools used to determine the transmission and absorption properties of materials. Typical spectrophotometers will produce spectra from 200 to 800 nm although different ranges are available. A spectrophotometer with a range of 200 to 800 nm is referred to as a UV/VIS spectrophotometer. There are also IR units. These devices may have a single light source or several light sources to radiate the material with the desired range of wavelengths. An LED light source could be provided to supply all or a portion of the required radiation. The LED light source could include an array of LEDs covering a wide spectral region including the ultraviolet and the infrared. The various wavelength LEDs could be independently controlled to provide specific wavelengths during the testing procedure. This method can reduce the amount of interference within the unit and as a result reduce the measurement error.

An ultraviolet or blue LED device according to the principles of the present invention may be used as a bug light to attract insects to be trapped or electrocuted. Insect control devices are typically constructed with fluorescent lamps and in some applications carbon dioxide emitters are also used. Insects have a photopic response curve that is sensitive to blue and ultraviolet light. They are also attracted to carbon dioxide. As a result, there are two kinds of bug killing devices predominantly used today: blue or black fluorescent light and carbon dioxide devices. These devices are designed to attract the insects and then kill them by electricity or physically trapping them. The fluorescent lamp can be replaced with the LED device and can be tailored to the particular insects' photopic response or the physical surroundings in which the device is used. The LED is a coherent light source, emitting light over a narrow wavelength range, and can be arranged to provide ultraviolet or deep blue light without providing visible light or if visible light is desirable, visible LEDs can be provided. A phosphor may be added to the LED or LED package to broaden the spectral emission if desired.

A bird's photopic response also includes the near ultraviolet region. As a result of seeing in the ultraviolet, objects may appear quite different to birds with many objects fluorescing. Birds may also use the ultraviolet to help them navigate. Birds, like many mammals and reptiles, also need ultraviolet light to produce vitamin D and without exposure to ultraviolet light they will suffer a variety of calcium deficient maladies. See www.users.mis.net/˜pthrush/lighting/uvmyth.html The Ultraviolet Myth: Lighting and Proper Diet by Patrick R. Thrush, 1999. LED lights as described herein can be provided to aid the health of birds kept in captivity as well as be used to deter them from certain areas.

There are many examples where full spectrum lighting was used to improve the lives of birds; one such study was conducted on chickens. In the past, chicken farms allowed chickens to be grown in coops with windows and access to the outdoors. The modern day chicken coop is now a poorly lit windowless building. Chickens were very productive in the outdoor coops as measured by their egg laying output as well as useful egg production years. Chickens were typically profitably productive for five years in these coops. In contrast, hens grown in the new windowless environments only last for 13 months. An experiment conducted by Dr. Ott showed that if full spectrum lighting with ultraviolet was used in the new chicken coop, the chicken's peak production lasted 3 years or more. The study also showed the birds ate $19,700 less feed per 50,000 chickens, laid 8.5% more eggs, cracked 2% fewer eggs, while laying larger eggs. Further, the birds did not need to be debeaked, because there was no cannibalism. This calculated into a total of $91,300 more profit for the farmer. See www.users.mis.net/˜pthrush/lighting/ott.html, Plain Common Sense vs. Scientific Theoretical Irrationality, By Dr. John N. Ott, also appeared in the International Journal of Biosocial Research, Special Subject Issue Volume 7, 1985.

As a result of experiments like those conducted by Dr. Ott, we can see that full spectrum lighting is not only healthy for humans but birds, reptiles, and other animals as well. A full spectrum or partial spectrum light made with LEDs can be provided for these applications as well as for human habitats. A full spectrum lighting device according to the present invention may be used for these applications and can be fixed on a particular color or the color can change with respect to time or other indicator. The amount of ultraviolet or IR can change throughout the day to simulate natural lighting conditions.

An ultraviolet lighting system according to the principles of the present invention may also be used to deter birds from living or feeding in certain areas. Birds are generally considered a hazard around airports because they can fly into the planes' path causing damage to the aircraft and death to the bird. The impact of a bird striking a high-speed aircraft can be dramatic when one considers that an aircraft flying at 500 kts. striking a large bird suffers an impact of nearly 1,500,000 ft.lbs. of energy. See http://www.tc.gc.ca/aviation/aerodrme/birdstke/info/hazard.htm, Bird Hazards, Transportation of Canada. To alleviate the problems associated with bird strikes, investigators have been searching for new methods of keeping birds away from aircraft. One such method is to use ultraviolet light in the area where the birds are a hazard.

Birds can see ultraviolet light and use it for vision and navigation. It is not understood if the deterring effect of the ultraviolet light is from the way things appear under the artificial irradiation or if it interferes with their navigation system. The ultraviolet devices could be set up in the airport as ground coverage lighting or the lighting could be used in the aircraft itself. The lighting could be irradiating in a constant direction or be movable. A beacon arrangement could also be used. Pulsing or wavelength shifting can easily be achieved with the LED based lighting device and this may serve as another method of deterring the birds. An ultraviolet light flashing in the area may annoy the birds, but the human occupation would not notice or be bothered by the invisible light show. The lighting device could also be used with other devices such as audio devices to provide noise with the lighting effects.

In another embodiment, the output of the LED(s) in a device may be controlled through an external signal such as that provided from a sensor, transducer, user interface or other signal generator. The signal generator may communicate a signal to a processor, or other circuit designed to receive the external signal and generate and/or communicate LED control signals in response thereto. The user interface may be of any type, e.g., a button, switch, dial or the like or it may be software controlled such that a computing device may be used to generate an external signal to control the output of the LED(s). It should be appreciated that there are many user interfaces and other signal generators that may be used to provide external signals to a device according to the principles of the present invention, and the present invention is not limited to use with any particular type of user interface or signal generator.

As used herein the term “ultraviolet” or “ultraviolet light” shall include the ultraviolet spectrum and the deep blue region of the visible spectrum.

As used herein the term the term “LED” should be understood to include light emitting diodes of all types, light emitting polymers, semiconductor dies that produce light in response to current, organic LEDs, electro-luminescent strips, and other such systems. An “LED” may refer to a single light emitting diode having multiple semiconductor dies that are individually controlled. It should also be understood that the term “LED” does not restrict the package type of the LED. The term “LED” includes packaged LEDs, non-packaged LEDs, surface mount LEDs, chip on board LEDs and LEDs of all other configurations. The term “LED” also includes LEDs packaged or associated with material (e.g., a phosphor) wherein the material may convert energy from the LED to a different wavelength.

While the invention has been disclosed in connection with the embodiments shown and described in detail, various equivalents, modifications, and improvements will be apparent to one of ordinary skill in the art from the above description. Such equivalents, modifications, and improvements are intended to be encompassed by the following claims.

1. A method, comprising:
A) generating ultraviolet radiation from at least one first LED;
B) irradiating an object with the ultraviolet radiation; and
C) controlling the ultraviolet radiation so as to generate at least one perceivable visual effect based on an interaction between the ultraviolet radiation and the object.

2. The method of claim 1, wherein the at least one object includes an ultraviolet-sensitive material disposed over at least one surface thereof that emits visible light when affected by the ultraviolet radiation.

3. The method of claim 2, wherein the at least one ultraviolet-sensitive material is arranged as a pattern over the at least one surface of the object.

4. The method of claim 2, wherein the at least one object is selected from the group consisting of: a retail display, a sign, an advertisement, a billboard, a logo, a picture, a graphical image, and a poster.

5. The method of claim 2, wherein the at least one object comprises at least one floor tile.

6. The method of claim 2, wherein the at least one object is disposed in an entertainment or amusement venue.

7. The method of claim 1, further comprising moving or rotating the at least one surface during the act C).

8. The method of claim 1, wherein the act C) comprises:
dynamically varying an intensity of the ultraviolet radiation.

9. The method of claim 8, wherein the act C) comprises:
flashing or pulsing the ultraviolet radiation.

10. The method of claim 9, further comprising moving or rotating the at least one surface of the object during the act C).

11. The method of claim 1, wherein the act C) comprises:
controlling the ultraviolet radiation so as to simulate outdoor conditions corresponding to different hours of a day.

12. The method of claim 1, further comprising:
D) generating visible radiation from at least one second LED,
wherein the act B) comprises irradiating the object with the ultraviolet radiation and the visible radiation, and
wherein the act C) comprises:
C1) controlling the at least one of the ultraviolet radiation and the visible radiation so as to generate the at least one effect based on an interaction between the at least one of the ultraviolet radiation and the visible radiation, and the object.

13. The method of claim 12, wherein the act C1) comprises:
C2) independently controlling a first intensity of the ultraviolet radiation and a second intensity of the visible radiation.

14. The method of claim 13, wherein the act C2) comprises:
independently controlling the first intensity of the ultraviolet radiation and the second intensity of the visible radiation in response to at least one addressed control signal.

15. An apparatus, comprising:
at least one first LED configured to generate ultraviolet radiation; and
at least one controller to control the at least one first LED so as to generate at least one perceivable visual effect based on an interaction between the ultraviolet radiation and an object irradiated by the ultraviolet radiation, the object comprising an ultraviolet-sensitive material disposed over at least one surface thereof that emits visible light when affected by the ultraviolet radiation.

16. The apparatus of claim 15, wherein the at least one controller is configured to control the at least one first LED to dynamically vary an intensity of the ultraviolet radiation.

17. The apparatus of claim 16, wherein the at least one controller is configured to control the at least one first LED to flash or pulse the ultraviolet radiation thereby substantially instantaneously creating a visual effect based on the interaction between the ultraviolet radiation and the object.

18. The apparatus of claim 15, further comprising:
at least one second LED configured to generate visible radiation,
wherein the at least one controller is configured to control at least one of the at least one first LED and the at least one second LED so as to generate the at least one effect based on an interaction between the ultraviolet radiation and the visible radiation, and the at least one object.

19. The apparatus of claim 18, wherein the at least one controller is configured to independently control a first intensity of the ultraviolet radiation and a second intensity of the visible radiation.

20. The apparatus of claim 19, wherein the at least one controller includes at least one addressable controller configured to independently control the first intensity of the ultraviolet radiation and the second intensity of the visible radiation in response to at least one addressed control signal.

21. An apparatus, comprising:
at least one first LED configured to generate ultraviolet radiation having a first spectrum; and
at least one addressable controller coupled to the at least one LED and configured to receive and process instructions to control the at least one LED based at least in part on an address associated with the at least one addressable controller.

22. The apparatus of claim 21, in combination with at least one surface, wherein the ultraviolet radiation, when generated, irradiates at least a portion of the at least one surface.

23. The apparatus of claim 22, wherein the surface includes at least one of a retail display, a sign, an advertisement, a billboard, a logo, a picture, a graphical image, and a poster.

24. The apparatus of claim 21, further comprising:
at least one second LED configured to generate visible radiation having a second spectrum different than the first spectrum,
wherein the at least one addressable controller is configured to independently control a first intensity of the ultraviolet radiation and a second intensity of the visible radiation based on the received instructions.

25. The apparatus of claim 24, further comprising:
at least one third LED configured to generate infrared radiation having a third spectrum different than the first spectrum,
wherein the at least one addressable controller is configured to independently control the first intensity of the ultraviolet radiation, the second intensity of the visible radiation, and a third intensity of the infrared radiation based on the received instructions.

26. The apparatus of claim 21, further comprising:
at least one third LED configured to generate infrared radiation having a third spectrum different than the first spectrum,
wherein the at least one addressable controller is configured to independently control the first intensity of the ultraviolet radiation and a third intensity of the infrared radiation based on the received instructions.
